(19) United States
(12) Patent Application Publication
(10) Pub. No.: US 2025/0381347 A1
(43) Pub. Date: Dec. 18, 2025

(54) WIRELESS ADJUSTABLE INJECTION AID AND METHOD OF USE THEREOF

(71) Applicant: YOUNG CHUL LEE, Busan (KR)

(72) Inventor: YOUNG CHUL LEE, Busan (KR)

(21) Appl. No.: 19/098,946

(22) Filed: Apr. 2, 2025

(30) Foreign Application Priority Data
Jun. 17, 2024 (KR) 10-2024-0078107

(51) Int. Cl. A61M 5/315; A61M 5/145; A61M 5/31

(52) U.S. Cl. CPC A61M 5/31568; A61M 5/1456; A61M 5/1458; A61M 2005/3114; A61M 2005/31588; A61M 2205/0216; A61M 2205/3592; A61M 2205/502; A61M 2205/584; A61M 2205/587

(57) ABSTRACT

The present invention discloses a wireless adjustable injection aid and method of use thereof. The device includes follows: 1st coupling unit coupled with the body unit of syringe, 2nd coupling unit coupled with piston of syringe, seating unit where the syringe coupled with the 1st and 2nd coupling units is settled, piston control unit that moves back and forth inside and outside the syringe body unit to control the back-and-forth movement of piston for drawing in or discharging fluid, and actuator electrically coupled with piston control unit to instruct operation of the piston control unit.

### TECHNICAL FIELD

The present disclosure relates to a wireless adjustable injection aid and its method of use. More specifically, the present disclosure is concerned with a wireless adjustable injection aid that inserts a needle inside the human body to inject medication or to aspirate body fluids, etc., reversely, and capable of controlling the piston of syringe using a linear actuator, and method of use thereof.

### BACKGROUND ART

The present disclosure enables a precise injection of medication into the body of patient by a practitioner (doctor, nurse) using a syringe. This is crucial for maximizing the effectiveness of treatment and reducing patient discomfort.

The present disclosure also reduces fear of error and mental burden experienced by a practitioner. This is crucial for preventing an unexpected accident caused by psychological stress, practice of practitioner, or patient conditions during injections performed in medical institutions.

In conventional injection methods, a syringe is filled with certain amount of medical solution in advance, hand-held in right or left hand, and the other hand is used to disinfect the injection site. Then, syringe needle is inserted at the injection site, and the practitioner uses the other hand, not holding the syringe, to pull the piston arm of syringe. Then, the practitioner checks the correct placement of needle and injects the medical solution. These multiple actions may cause errors, due to shaking of syringe between the steps. This leads to difficulty in performing a meticulous or precise injection.

Recently, clinical medicine field has developed and utilized various diagnostic and treatment instruments for patient disease diagnosis, operation, prognostic management, disease prevention, and health promotion. If a practitioner performs a syringe injection while using one hand for ultrasound, etc., this may also lead to difficulty in performing a meticulous or precise injection.

The effectiveness of the injection method depends on the skill level of the practitioner, which inherently involves the risk of medical accidents. Any unexpected medical accident may cause severe additional pain to the patient, or even death, in extreme cases, causing significant psychological distress to the practitioner. Such accidents may also expand to medical disputes.

The present disclosure is proposed as a concrete measure is required to solve the problem, and it is necessary to develop an instrument to aid injection while maintaining a consistent syringe pressure. In response, the present disclosure was invented to address the existing problems.

### SUMMARY

The objective of the present disclosure is to provide a wireless adjustable injection aid, which uses a detachable syringe needle in the area of wireless adjustable injection aid in order to eliminate the need for excessive force during the injection and prevent any shaking during the procedure.

The wireless adjustable injection aid of the present disclosure includes 1st coupling unit connected to syringe body, 2nd coupling unit connected to the syringe piston, seating unit for positioning the syringe connected with 1st and 2nd coupling unit, piston control unit for controlling back-and-forth movement of piston that moves inside and outside the syringe for fluid aspiration or discharge, and actuator electrically connected to the piston control unit for operation of piston control unit.

Fluid may be drawn into the syringe body through the fluid line or may be discharged outside the syringe body when the piston control unit is operated through the needle coupling unit included in the actuator binding syringe needle separated from the syringe, fluid line connecting the syringe needle to the discharge unit of the syringe, and actuator.

The actuator includes the 1st switch and 2nd switch. When 1st switch is turned on, fluid inside the syringe is discharged outside the syringe. When 2nd switch is turned on, fluid may be drawn into the syringe.

The 2nd coupling unit includes 1st member and 2nd member. The 2nd coupling unit may also include 1st groove unit, which is formed in 1st member, that settles a unit of outer peripheral surface of the syringe's pressing unit as well as 2nd groove unit, which is formed in 2nd member, that settles another unit of outer peripheral surface of the syringe's pressing unit.

The present disclosure may also include an injection aid system, which includes wireless adjustable injection aid, syringe, and fluid line.

The methods of using the wireless adjustable injection aid in the present disclosure are as follows: Separate a syringe needle from the syringe. Couple the separated syringe to the 1st and 2nd coupling units of the wireless adjustable injection aid, connect with the wireless adjustable injection aid, and connect piston included in the syringe and wireless adjustable injection aid's piston control unit. Couple the syringe needle to the actuator electrically connected to the piston control unit. Use fluid line to couple the syringe needle and discharge unit included in the syringe. Use the actuator to operate the piston control unit, control piston movement, and discharge fluid inside the syringe.

The step of using the actuator to operate the piston control unit, control the piston movement, and discharge the fluid inside the syringe can be replaced with the step of using the actuator to operate the piston control unit, control the piston movement, and draw the fluid inside the syringe.

### ADVANTAGEOUS EFFECTS

The wireless adjustable injection aid and method of use thereof for the present disclosure enables attachment and detachment of the aid at once without structural modifications for syringes of different sizes.

The present disclosure includes the wireless adjustable injection aid, which allows users control suction and injection without directly holding the syringe body, to protect the hands of user, prevent use of excessive force, and enable injection, suction, and aspiration of specific amount without shaking.

The present disclosure also enables precise and accurate injection when a user is already using one hand for medical practices, such as ultrasound, and performing injection additionally.

The effects of the present disclosure are not limited to the details described above, and more effects are included in the specification.

### BRIEF DESCRIPTION OF THE DRAWINGS

For better understanding of embodiments of the specification, refer to the following description for the attached drawings with referential remarks indicating the same or functionally similar elements.

FIG. 1 is a conceptual diagram of a wireless adjustable injection aid system for embodiment of the present disclosure.

FIG. 2 is a drawing of a wireless adjustable injection aid for embodiment of the present disclosure.

FIG. 3 shows a wireless adjustable injection aid alongside a syringe for embodiment of the present disclosure.

FIG. 4 is a drawing of the structure of a wireless adjustable injection aid for embodiment of the present disclosure.

FIG. 5 is a drawing of the principle of syringe insertion into the wireless adjustable injection aid for embodiment of the present disclosure.

FIG. 6 is a drawing of an actuator of the wireless adjustable injection aid for embodiment of the present disclosure.

FIG. 7 is a drawing of the power-supplied actuator of FIG. 6.

FIG. 8 is an operational diagram showing the process of injecting liquid using a wireless adjustable injection aid for embodiment of the present disclosure.

FIG. 9 is an operational diagram of the process of liquid aspiration using a wireless adjustable injection aid for embodiment of the present disclosure.

FIG. 10 is a flowchart of the usage method of a wireless adjustable injection aid for embodiment of the present disclosure.

FIG. 11 is a flowchart of the usage method of a wireless adjustable injection aid for another embodiment of the present disclosure.

FIG. 12 is a drawing of a wireless adjustable injection aid operated by a smart device for embodiment of the present disclosure.

The referenced figures are not necessarily illustrated to scale and shall be considered as the simplified representation of various preferred features that exemplify the fundamental principles of the present disclosure. For example, certain design features of the present disclosure, including specific dimensions, direction, positions, and shapes, may be partially determined by the intended application and usage environment.

### DETAILED DESCRIPTION

In the present disclosure, the term “fluid” refers to the medicinal fluid drawn into the syringe or discharged from the syringe, regardless of types of fluid. The embodiments in drawings describe the present disclosure in more details.

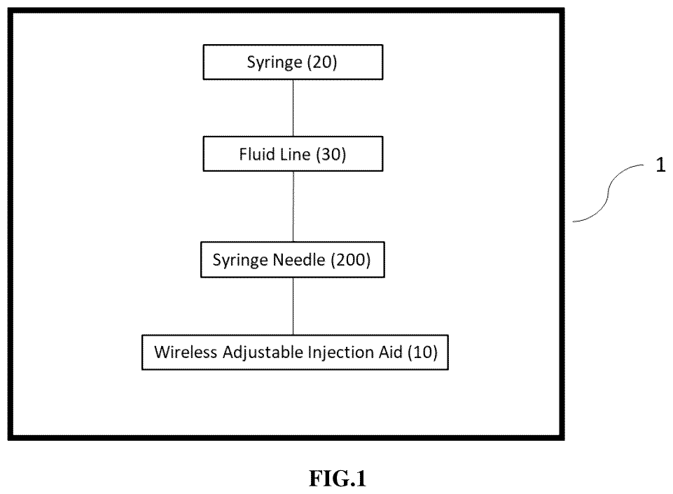

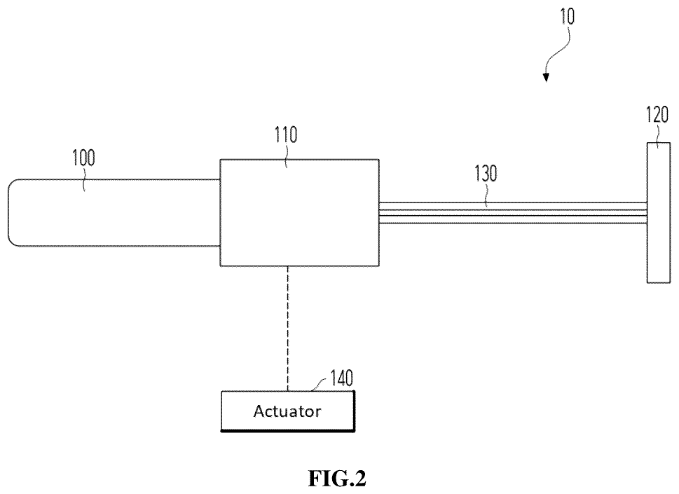

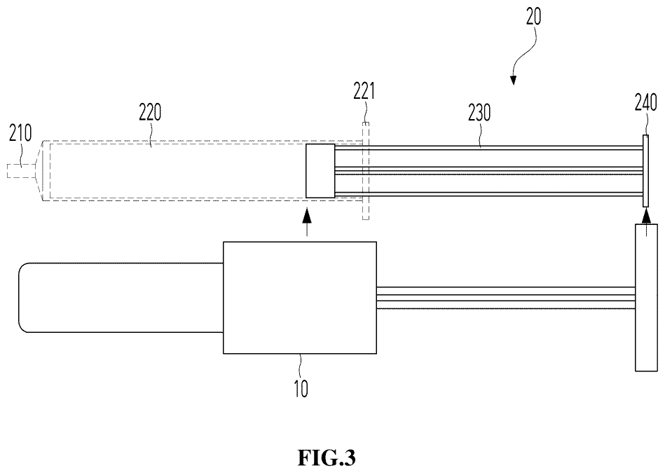

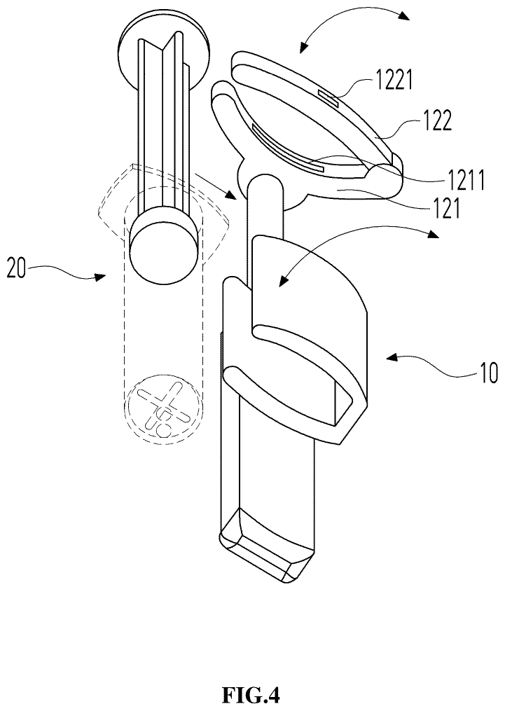

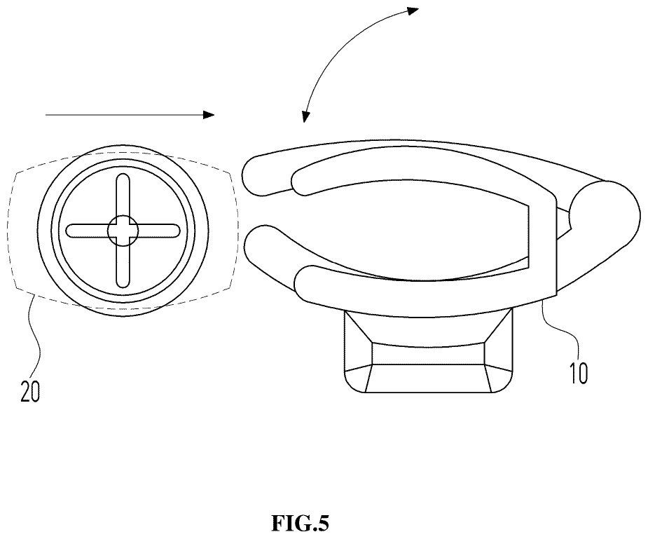

FIG. 1 is a conceptual diagram of a wireless adjustable injection aid system for embodiment of the present disclosure. FIG. 2 is a drawing of a wireless adjustable injection aid for embodiment of the present disclosure. FIG. 3 is a drawing of a wireless adjustable injection aid alongside a syringe for embodiment of the present disclosure. FIG. 4 is a drawing of the structure of a wireless adjustable injection aid for embodiment of the present disclosure. FIG. 5 is a drawing of the principle of syringe insertion into the wireless adjustable injection aid for embodiment of the present disclosure.

The injection aid system (1) of the present disclosure primarily includes a wireless adjustable injection aid (10), a syringe (20), and a fluid line (30). The syringe (20) may be coupled with a needle (200) and includes a discharge unit (210) with a hole for injection and discharge of fluid inside and outside the syringe, a body unit (220) that stores the infusion fluid, a piston (230) that enables back-and-forth movement inside and outside the body unit (220), a wing unit (221) at one end of the body unit to serve as the starting point for back-and-forth movement of piston (230), and a pressing unit (240) that pulls and pushes piston (230).

Needle (200) is detachable from the syringe (20). Specifically, needle (200) can be coupled with or separated from discharge unit (210). Body unit (220) has a cylindrical shape for safe storage of fluid. Thus, body unit (220) functions as a cylinder for storage of fluid, etc.

Outer radius of wing unit (221) is larger than the outer radius of body unit (220). Pressing unit (240) may have a disc shape for easier pressing. Its structure can be grasped by the user for manual pressing, and the shape is not partially limited.

The wireless adjustable injection aid (10) for embodiment of the present disclosure may include 1st coupling unit (110) connected to body unit (220) of the syringe (20). The 1st coupling unit (110) may have a hollow structure to cover body unit (220). This allows body unit (220) to slide into the 1st coupling unit (110) for coupling.

The wireless adjustable injection aid (10) for embodiment of the present disclosure may include 2nd coupling unit (120) connected to the piston (230) of the syringe (20). The 2nd coupling unit (120) can be interconnected with piston (230) by grasping the piston (230). The 2nd coupling unit (120) can be coupled with the pressing unit (240) formed at one end of the piston (230). The 2nd coupling unit (120) can be coupled with pressing unit (240) by grasping pressing unit (240).

In summary, as the 1st coupling unit (110) is coupled with body unit (220), the 2nd coupling unit (120) is coupled with piston (230), and syringe (20) is settled in seating unit (100) of the wireless adjustable injection aid (10), the syringe (20) can be fixed stably to the aid (10).

In one embodiment, the 1st coupling unit (110) and the 2nd coupling unit (120) may be made of elastic material, allowing for various sizes and capacities of the syringes (20). The units can be attached and detached to syringes (20) of different sizes and capacities, such as syringes (20) with 10 ml to 50 ml capacity, without structural modification.

In one embodiment, the 2nd coupling unit (120) may be divided into the 1st member (121) and the 2nd member (122). In one embodiment, the 1st member (121) and the 2nd member (122) may be coupled with a hinge at one end of each member.

The 1st member (121) may form 1st groove unit (1211) for partial coupling of the outer surface of the syringe's pressing unit (240). The 2nd member (122) may form 2nd groove (1221) for coupling of another unit of the outer surface of the syringe's pressing unit (240).

As the 2nd coupling unit (120) and the pressing unit (240) can be fixed through the grooves, the syringe (20) may remain attached to the wireless adjustable injection aid (10) during the piston movement.

The wireless adjustable injection aid (10) for the embodiment of the present disclosure may include a piston control unit (130) that controls the back-and-forth movement of the piston (230), which moves within the body unit (220) of the syringe (20) to draw in or discharge the fluid.

In one embodiment, piston control unit (130) can be formed between the 1st coupling unit (110) and the 2nd coupling unit (120) and may have a rod shape that interconnects the 1st coupling unit (110) and the 2nd coupling unit (120).

Thus, the 1st coupling unit (110) and the 2nd coupling unit (120) hold the syringe (20) at both ends of the piston control unit (130).

In one embodiment, the length of piston control unit (130) can be extended and reduced in the forward and backward directions. In another embodiment, the length of piston control unit (130) can be shortened by being drawn into the seating unit (100), or can be extended by getting out of the seating unit (100). The extension and contraction of the piston control unit (130) can be controlled by an actuator (140), to be described later.

The wireless adjustable injection aid (10) for embodiment of the present disclosure may include an actuator (140) electrically connected to the piston control unit (130) to instruct operation of the piston control unit (130).

When the actuator (140) activates the piston control unit (130), the piston control unit (130) is coupled with the 1st coupling unit (110) and the 2nd coupling unit (120) while the 2nd coupling unit (120) is coupled with the piston (230). As the piston control unit (130) extends or contracts, the piston (230) can move into or move out of the body unit (220) of the syringe (20).

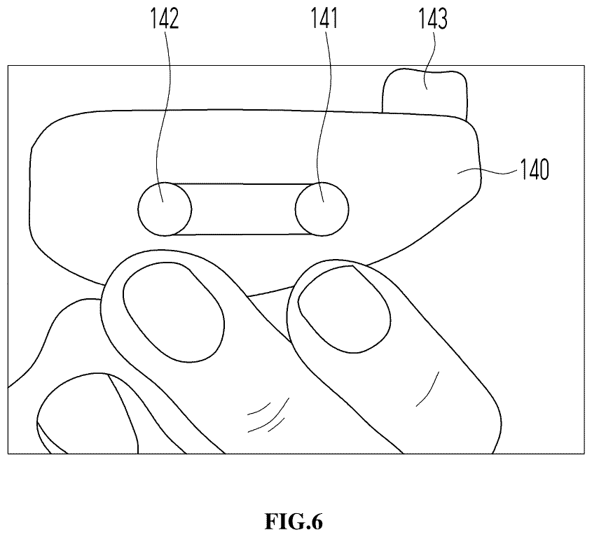

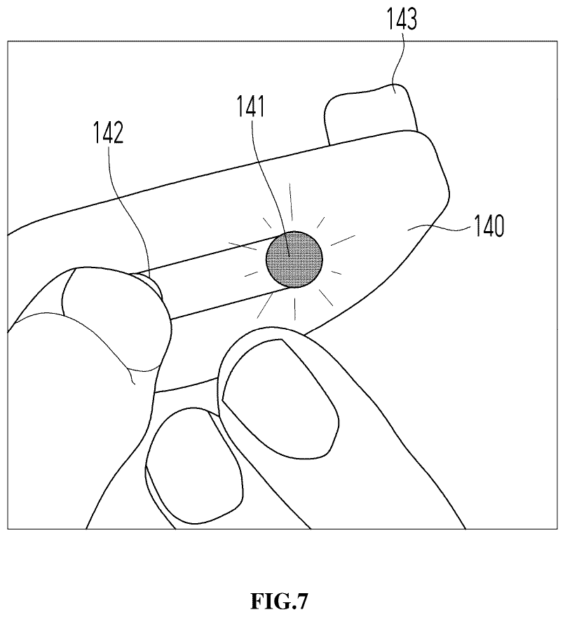

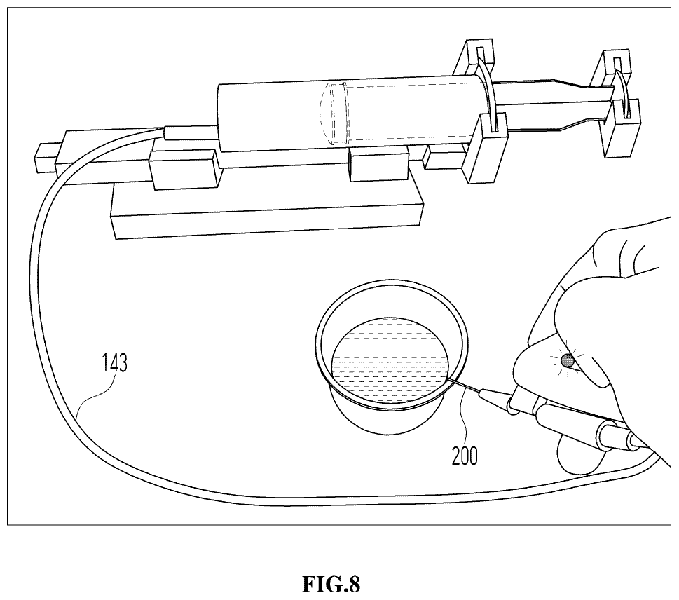

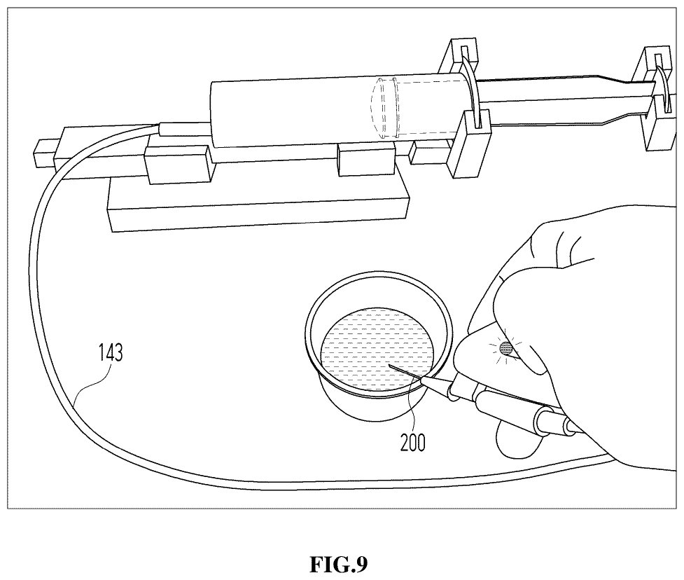

FIG. 6 is a drawing of the actuator of the wireless adjustable injection aid for embodiment of the present disclosure. FIG. 7 is a drawing of the power-supplied actuator of FIG. 6. FIG. 8 is an operational diagram illustrating the process of using the wireless adjustable injection aid for embodiment of the present disclosure to inject fluid. FIG. 9 is an operational diagram illustrating the process of using the wireless adjustable injection aid for embodiment of the present disclosure to aspirate fluid.

As shown in FIGS. 6 to FIG. 9, the actuator (140) for the present disclosure may include a needle coupling unit (143) that allows multiple needles, including a needle (200) detached from the syringe (20), to be coupled.

The needle (200) or multiple needles are coupled with one or more fluid lines (30) that connect the discharge unit (210) of the syringe (20). When the actuator (140) is used to operate the piston control unit (130) and the piston (230) is pushed out of the body unit (220), fluid is drawn in through the needle (200) and guided through the fluid line (30) into the syringe body (220). Reversely, when the piston (230) moves into the body unit (220), fluid can be expelled from the syringe body (220).

The discharged fluid travels through the fluid line (30) and reaches the syringe needle (200). It is also discharged externally through the syringe needle. In one embodiment, the fluid line (30) may be made of a transparent material to observe the fluid flow, but it is not limited to this material.

The actuator (140) of the present disclosure may include 1st switch (141) and 2nd switch (142). In one embodiment, when the 1st switch (141) is turned on, the fluid inside the syringe (20) can be discharged externally. When the 2nd switch (142) is turned on, the fluid can be drawn into the syringe (20).

Thus, the 1st switch (141) and the 2nd switch (142) can instruct the piston control unit (130) to move in opposite directions from the back-and-forth axis.

In one embodiment, each switch of the actuator (140) may include a light source. When a switch is turned on, the light source emits light, so that the user can visually check the operation of the wireless adjustable injection aid (10).

In another embodiment, color of the light source may vary depending on whether the fluid is being drawn into the syringe (20) or discharged from it. Thus, the light sources in the 1st switch (141) and the 2nd switch (142) may have different colors, so that the user can intuitively verify whether the aid is operating as intended or commanded.

In one embodiment, the actuator (140) may include multiple needle-coupling units (143). This allows coupling of multiple syringes (20) with different needles (200). Each syringe needle (200) can be connected to the discharge unit (210) of the corresponding syringe (20) through respective fluid line (30).

Thus, when the actuator (140) is operated, it can fill fluids to all connected syringes (20) or discharge fluid from each syringe (20) at the same time.

In summary, the wireless adjustable injection aid (10) controls injection and discharge by using fluid line (30) to connect the discharge unit (210) of the syringe and couple needles, instead of directly grasping the body unit (220) of the syringe (20). This prevents users from using excessive force and allows injecting and discharging specific amount of fluid stably. This also protects the hand of a practitioner (medical personnel).

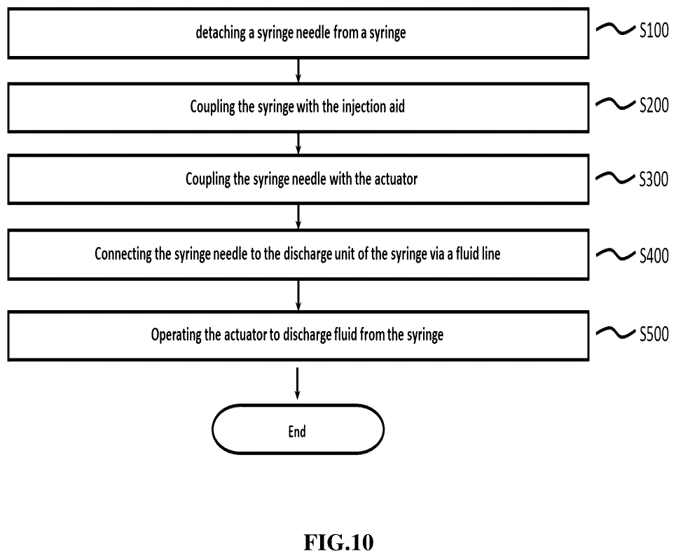

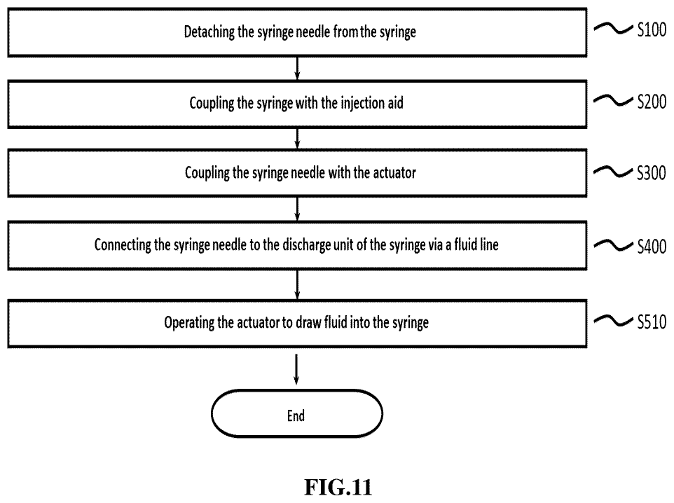

FIG. 10 is a flowchart on the method of using the wireless adjustable injection aid according to embodiment of the present disclosure. FIG. 11 is a flowchart on the method of using the wireless adjustable injection aid for another embodiment of the present disclosure.

The present disclosure can be implemented as a process invention for method of using the wireless adjustable injection aid. The method for using the wireless adjustable injection aid includes the following sequential steps: (S100) Detach the syringe needle (200) from the syringe (20). (S200) Couple the syringe (20) detached of syringe needle (200) and couple with 1st member (110) and 2nd member (120) of the wireless adjustable injection aid (10) for coupling piston (230) included in the syringe (20) with the piston control unit (130) of the wireless adjustable injection aid (10). (S300) Couple the syringe needle (200) to the actuator (140) electrically connected to the piston control unit (130). (S400) Couple the syringe needle (200) and the discharge unit (210) of the syringe (20) to fluid line (30). (S500) Use actuator (140) to operate the piston control unit and control the movement of the piston (230) and discharge the fluid inside the syringe (20).

The step (S500) of using the actuator (140) to operate the piston control unit (130), control the movement of piston (230), and discharge fluid inside the syringe (20) can be changed to the step (S510) of using the actuator (140) to operate the piston control unit (130), control the movement of piston (230), and draw fluid inside the syringe (20).

Thus, the wireless adjustable injection aid (10) allows discharge of fluid from the syringe (20) and the injection of fluid into the syringe (20).

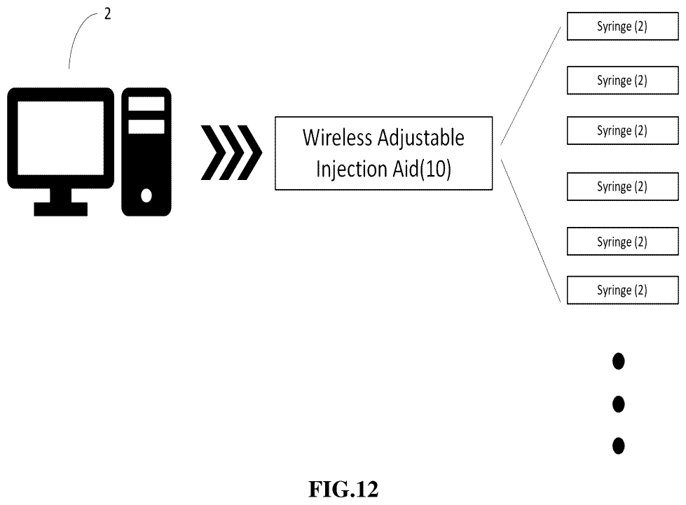

FIG. 12 is a drawing of a wireless adjustable injection aid being operated by a smart device for one embodiment of the present disclosure.

The wireless adjustable injection aid (10) for the present disclosure allows integrated control and management by a smart device (2) to comprehensively control the process of discharging fluid from multiple syringes (20) and drawing fluid into the syringes (20).

The smart device (2) can adjust the speed and volume of the fluid being drawn into or discharged from each syringe respectively (20). Accordingly, the wireless adjustable injection aid (10) may include multiple actuators (140).

The smart device (2) for embodiment of the present disclosure may include general computers, such as desktop computers and laptops, and mobile devices, such as smartphones, tablet PCs, PDAs (Personal Digital Assistants), and mobile communication terminals. This also should be interpreted as all devices capable of computing.

The operating system of the smart device (2), where the application (3) is stored, may be operating systems, such as Windows or Macintosh, installed on general computers, such as like desktops and laptops. It also may be mobile operating systems, such as iOS or Android, installed on mobile devices, such as smartphones and tablet PCs.

The embodiment of the present disclosure may have a computer program that implements Caddy management system (1), and this computer program can be saved in a recording medium that can be read by using computers. To improve a mild cognitive impairment for the embodiment implemented by a computer program saved in recording medium and read by the computer, the computer program (20) may include code written in computer languages, such as C, C++, JAVA, or machine language, that processor (CPU) of computer can read. The code may include functional code related to functions defining the functions and may contain control code for the computer processor to implement the functions in a certain order.

The code may further include code related to memory reference on referencing the location (address) in the internal or external memory of the computer for additional information or media required for computer processors to implement the functions.

If communication with another remote computer, server, etc., is necessary for computer processor to implement the functions, the code may include codes related to communication on how the computer processor uses communication module (example: wired/wireless communication module) to communicate with another computer, server, etc., and how the computer processor transmits and receives media and information during communication.

The computer program, relevant code, code segments, etc., for implementing these embodiments may be easily inferred or modified by programmers of the relevant technical field of the present disclosure, considering the system environment of the computer that reads the recording medium to execute the program.

The recording medium that can be read by using the computer containing computer program is distributed to the computer system connected through network to save and execute the computer-readable code based on a distributed method. In this case, the execution results can be sent to one or more of distributed computers, and the computers that received such results can also execute some of the functions to send them to other distributed computers.

The description covers the technical concept of the present disclosure with embodiments. Those with general knowledge on the relevant technical field will be capable of modifying and altering while maintaining the fundamental characteristics of the present disclosure. Thus, the embodiments in the present disclosure are intended for description purpose rather than limiting the technical concept of the present disclosure. The embodiments also do not restrict the scope of the technical concept of the present disclosure. The scope of protection for the present disclosure shall be interpreted based on the following claim, and all technical concepts within the same scope should be interpreted as being included within the scope of right for the present disclosure.

### DESCRIPTION OF SYMBOLS

1: Injection Aid System
2: Smart Device
10: Wireless Adjustable Injection Aid
100: Seating Unit
110: 1st Coupling Unit
120: 2nd Coupling Unit
121: 1st Member
1211: 1st Groove Unit
122: 2nd Member
1221: 2nd Groove Unit
130: Piston Control Unit
140: Actuator
141: 1st Switch
142: 2nd Switch
143: Needle Coupling Unit
20: Syringe
200: Syringe Needle
210: Discharge Unit
220: Body Unit
221: Wing Unit
230: Piston
240: Pressing Unit
30: Fluid Line

1. A structure of a wireless adjustable injection aid, comprising:
a 1st coupling unit configured to cover and couple with a body unit of a syringe;
a 2nd coupling unit configured to interconnect with a piston of the syringe by grasping the piston;
a seating unit configured to mount the syringe, the seating unit being coupled with the 1st and 2nd coupling units;
a rod-shaped piston control unit having one end coupled with the 1st coupling unit and another end coupled with the 2nd coupling unit, the piston control unit being formed between the 1st and 2nd coupling units and configured to control the back-and-forth movement of the piston to draw in or discharge fluid by moving inside and outside the body unit of the syringe;
an actuator electrically coupled with the piston control unit to instruct operation of the piston control unit by making the piston control unit draw inside the seating unit to reduce length or get outside from the seating unit to extend length;
multiple needle coupling units formed on the actuator, each configured to couple with a respective syringe needle; and
multiple fluid lines having one end coupled with a respective syringe needle and the other end coupled with a respective discharge unit of syringe,
wherein the 1st and 2nd coupling units are made of elastic materials to allow coupling with syringes regardless of size and capacity,
wherein the actuator includes a 1st switch and a 2nd switch, and wherein activation of the 1st switch causes the piston control unit moves in 1st direction to discharge the fluid inside the syringe body unit coupled with the fluid lines, and activation of the 2nd switch causes the piston control unit moves in 2nd direction to draw fluid into the syringe body unit coupled with the fluid lines,
wherein the 1st switch and the 2nd switch each include a light source that emits light when the respective switch is operated, and each light source emits light in a different color to distinguish the switches during operation.

2. The structure of claim 1,
wherein the 2nd coupling unit comprises:
a 1st member; and
a 2nd member;
wherein the 1st member includes a 1st groove configured to receive and secure a portion of an outer peripheral surface of a pressing portion of the syringe; and
wherein the 2nd member includes a 2nd groove configured to receive and secure another portion of the outer peripheral surface of the pressing portion of the syringe.

3. A wireless adjustable injection aid system, comprising:
the wireless adjustable injection aid of claim 2;
a syringe; and
a fluid line.

4. A method of using the wireless adjustable injection aid of claim 1, comprising:
detaching a syringe needle from a syringe;
coupling the syringe, from which the syringe needle has been detached, to a 1st coupling unit and a 2nd coupling unit of the wireless adjustable injection aid to secure the syringe to the injection aid;
coupling the syringe needle with one of multiple needle coupling units formed on an actuator electrically connected to a piston control unit;
connecting the syringe needle and a discharge unit of the syringe using a fluid line; and
operating the actuator to instruct the piston control unit to control movement of the syringe piston to discharge fluid from inside the syringe.

5. The method of claim 4,
wherein the step of operating the actuator to instruct the piston control unit to control movement of the syringe piston to discharge fluid from inside the syringe is replaceable with a step of operating the actuator to instruct the piston control unit to control movement of the syringe piston to draw fluid into the syringe.
